(19) United States
(12) Patent Application Publication
(10) Pub. No.: US 2014/0114200 A1
(43) Pub. Date: Apr. 24, 2014

(54) COMPOSITE MANUAL AND AUTOMATED FETAL ANALYSIS SYSTEMS AND METHODS

(71) Applicant: Hill-Rom Services, Inc., Batesville, IN (US)

(72) Inventors: William B. Bishop, Apex, NC (US); Kyle Patrick, Cary, NC (US); Daleep Bhatia, Cary, NC (US)

(73) Assignee: Hill-Rom Services, Inc., Batesville, IN (US)

(21) Appl. No.: 14/056,165

(22) Filed: Oct. 17, 2013

Related U.S. Application Data
(60) Provisional application No. 61/715,896, filed on Oct. 19, 2012.

(51) Int. Cl. A61B 5/00; A61B 5/024

(52) U.S. Cl. CPC A61B 5/4362; A61B 5/02411
USPC 600/483

(57) ABSTRACT

A composite manual and automated fetal strip analysis system is disclosed. A computer is configured to receive at least one of a signal indicative of said physiological parameter of a fetus and a signal indicative of uterine activity and automatically detect a pre-determined pattern. A user interface allows a user to manually alter results of the automatic detection.

### CROSS-REFERENCE TO RELATED APPLICATIONS

The present application claims the benefit, under 35 U.S.C. §119(e), of U.S. Provisional Application No. 61/715,896, which was filed Oct. 19, 2012, and which is hereby incorporated by reference herein in its entirety.

### BACKGROUND

Analysis of the physiological parameters of a fetus and a mother to be is an ongoing challenge. While several systems and methods exist to analyze these parameters, opportunity exists for further development in this area.

### BRIEF SUMMARY

The present disclosure includes one or more of the features recited in the appended claims and/or the following features which, alone or in any combination, may comprise patentable subject matter.

One embodiment of a system may comprise a computer comprising a software program configured to normalize gaps in at least one of a signal indicative of a physiological parameter of a fetus and a signal indicative of uterine activity. A display whereon the software program is configured to display at least one of the signal indicative of the physiological parameter of a fetus and the signal indicative of uterine activity.

Another embodiment of a system may comprise a fetal monitoring system configured to capture at least one of a signal indicative of the physiological parameter of a fetus and a signal indicative of uterine activity. A computer configured to communicate with the fetal monitoring system, the computer comprising a software program configured to normalize gaps in at least one of a signal indicative of the physiological parameter of a fetus and a signal indicative of uterine activity. A display, whereon the software program is configured to display at least one of the signal indicative of the physiological parameter of the fetus and the signal indicative of the uterine activity. A user interface configured to communicate with the computer, the user interface configured to allow a user to initiate automatic detection of a pre-determined pattern from at least one of the signal indicative of the physiological parameter of a fetus and the signal indicative of uterine activity.

One embodiment of a system comprises means for initiating automatic detection of a pre-determined pattern from at least one of a signal indicative of said physiological parameter of a fetus and a signal indicative of uterine activity. Means for displaying results of said automatic detection of said pre-determined pattern. Means for allowing a user to manually alter said results of said automatic detection. Means for allowing a user to save said altered results of said automatic detection in an electronic database.

### BRIEF DESCRIPTION OF THE DRAWINGS

The accompanying drawings incorporated in and forming a part of the specification illustrate several aspects of the claimed subject matter and, together with the description, serve to explain the principles of the claimed subject matter. In the drawings:

FIG. 1 is a block diagram of a composite manual and automated fetal analysis system allowing a caregiver to choose a time segment for analysis, constructed according to one or more of the principles disclosed herein;

FIG. 2 is a block diagram of a composite manual and automated fetal analysis system allowing a caregiver to initiate detection of patterns, constructed according to one or more of the principles disclosed herein;

FIG. 3 is a block diagram of a composite manual and automated fetal analysis system allowing a caregiver to manually edit the displayed analysis patterns, constructed according to one or more of the principles disclosed herein;

FIG. 4 is a block diagram of a composite manual and automated fetal analysis system allowing a caregiver to save the resulting hybrid automatic/manual analysis as in entry in the patient's electronic medical record, constructed according to one or more of the principles disclosed herein;

FIG. 5 is a flowchart showing a method of use of a composite manual and automated fetal analysis system, constructed according to one or more of the principles disclosed herein; and

FIGS. 6A and 6B, together, form another flowchart showing an analysis performed by a composite manual and automated fetal analysis system to identify and correct artifacts in data, constructed according to one or more of the principles disclosed herein.

### DETAILED DESCRIPTION OF ILLUSTRATIVE EMBODIMENTS

The embodiments of the claimed subject matter and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be briefly mentioned or omitted so as to not unnecessarily obscure the embodiments of the claimed subject matter described. The examples used herein are intended merely to facilitate an understanding of ways in which the claimed subject matter may be practiced and to further enable those of skill in the art to practice the embodiments of the claimed subject matter described herein. Accordingly, the examples and embodiments herein are merely illustrative and should not be construed as limiting the scope of the claimed subject matter, which is defined solely by the appended claims and applicable law. Moreover, it is noted that like reference numerals represent similar parts throughout the several views of the drawings.

It is understood that the subject matter claimed is not limited to the particular methodology, protocols, devices, apparatus, materials, applications, etc., described herein, as these may vary. It is also to be understood that the terminology used herein is used for the purpose of describing particular embodiments only, and is not intended to limit the scope of the claimed subject matter.

Unless defined otherwise, all technical and scientific terms used herein have the same meanings as commonly understood by one of ordinary skill in the art.

The subject matter herein is directed to a composite manual and automatic fetal analysis systems and methods for use in hospitals and other health care settings where care for expecting mothers may be provided.

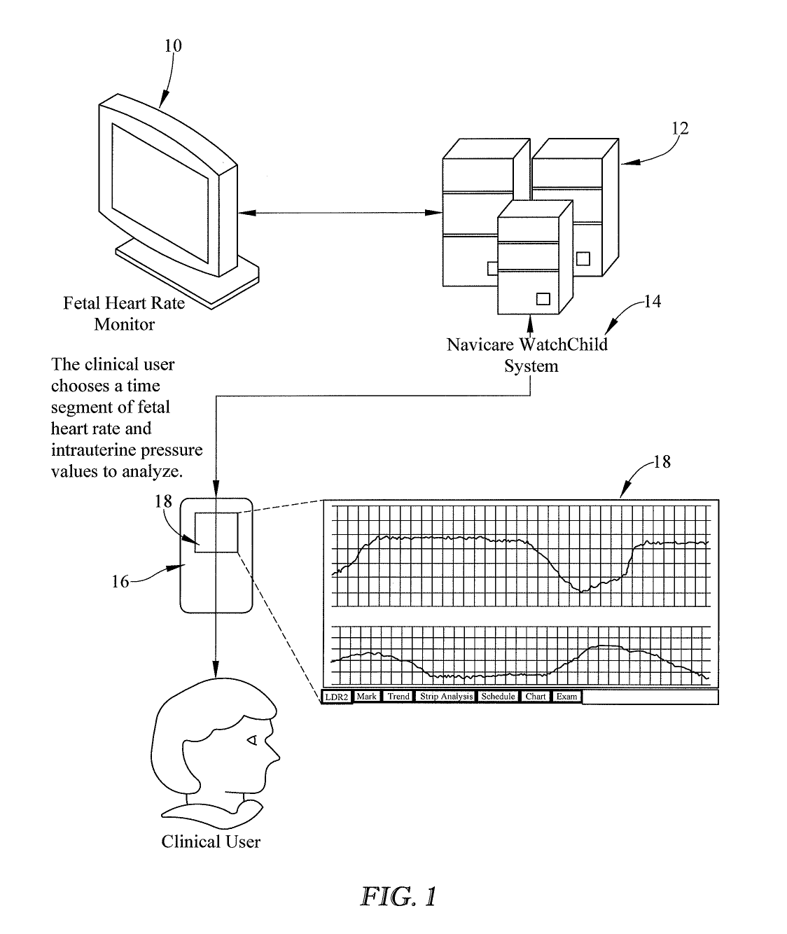

One illustrative embodiment of a composite manual and automatic fetal analysis system is shown in FIG. 1. A fetal heart rate monitor 10 is configured to communicate with a computer or a server 12. The fetal heart rate monitor 10 is any system or device capable of communicating at least one of said signal indicative of said physiological parameter of a fetus and said signal indicative of uterine activity. In this embodiment, the fetal heart rate monitor 10 communicates with the computer 12 via an electrical signal while in other embodiments, the fetal heart rate monitor 10 may communicate with the computer 12 using any other type of signal including but not limited to optical and acoustic signals. In one embodiment, the fetal heart monitor 10 is configured to measure and transmit to the computer 12 a signal indicative of the fetal heart rate and a signal indicative of the uterine pressure data. In other embodiments, the fetal heart rate monitor 10 is configured to transmit any combination of signals indicative of fetal heart rate and uterine pressure data to computer 12.

A software program 14 installed on the computer 12 is configured to receive data from the fetal heart rate monitor 10 in this application. In other embodiments, the software program 14 is a web based application which although resident on a computer 12 is accessed via another computer 12 and a computer network. In this embodiment, the software program is the NaviCare® WatchChild® solution. A user interface 16 allows a user to access the software program 14 and allows a user to provide inputs to the program. In this embodiment, the user interface 18 is a computer station and comprises a display screen 18. In other embodiments, the user interface 18 is a touch sensitive screen which also serves as the display 16. The display 18 is configured to show data representative of at least one signal received from the fetal heart rate monitor 10. As shown in FIG. 1, the composite manual and automatic fetal analysis system allows a user to select a time segment of signals received from the fetal heart rate monitor 12 for analysis.

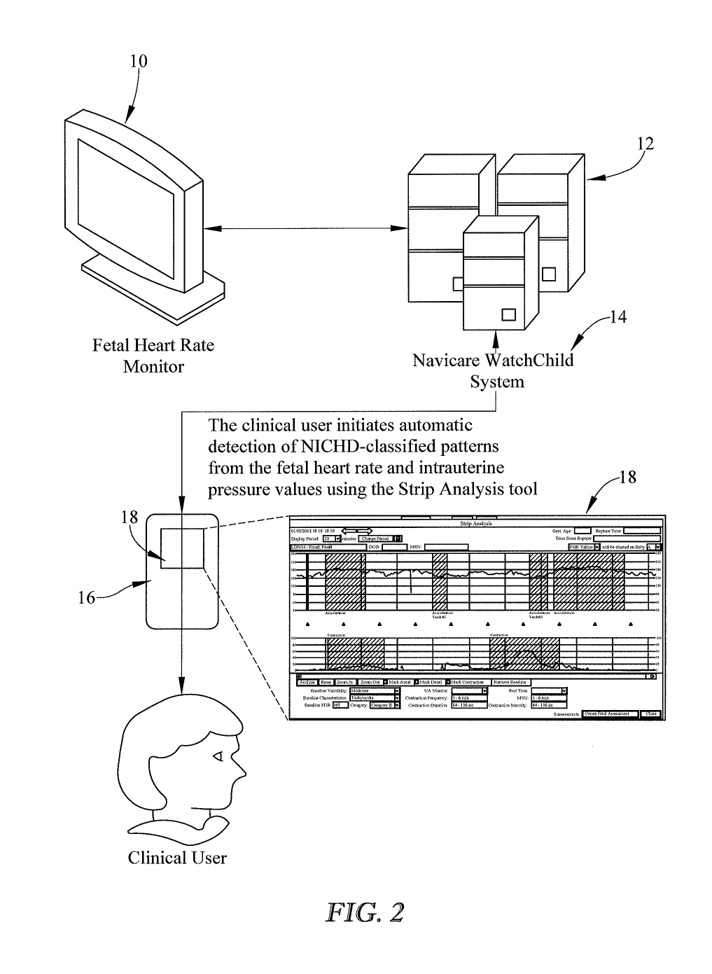

FIG. 2 shows the composite manual and automatic fetal analysis system of FIG. 1 wherein the user interface 16 allows a user to initiate automatic detection of predetermined patterns in a representative data set of data received from the fetal heart rate monitor 10. In this embodiment, a representative data set is created from the data received from the fetal heart rate monitor which is conditioned by elimination of undesirable spikes and interpolating gaps. In another embodiment, the automatic detection of predetermined patterns is performed on unconditioned data received from the fetal heart rate monitor 10. The software program 14 calculates the National Institute of Child Health and Human Development (NICHD) characteristics of the time segment selected by the user. These characteristics include at least one of, but are not limited to, fetal heart rate baseline, variability, classification, accelerations, decelerations, uterine contractions and features related to uterine contractions (intensity, frequency, duration, rest tone and Montevideo units). In this embodiment, the software program 14 is configured to identify pre-determined patterns based on National Institute of Child Health and Human Development (NICHD) guidelines and display patterns identified in the data as color overlays on the display 18. In this embodiment, the color overlays are displayed on top of fetal heart rate signal and uterine pressure data shown on display 18.

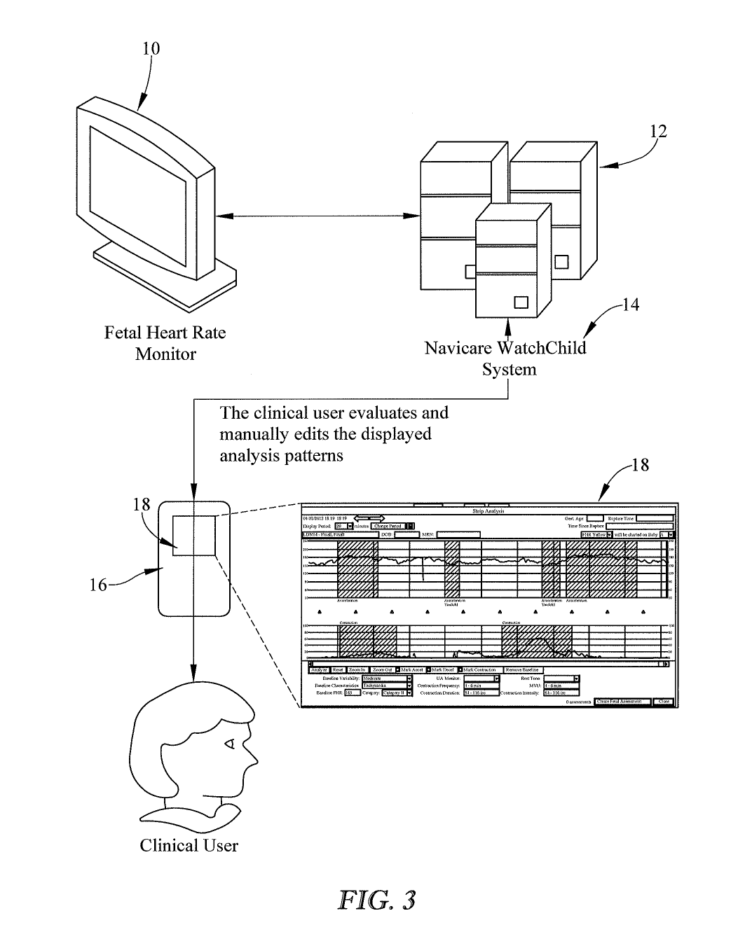

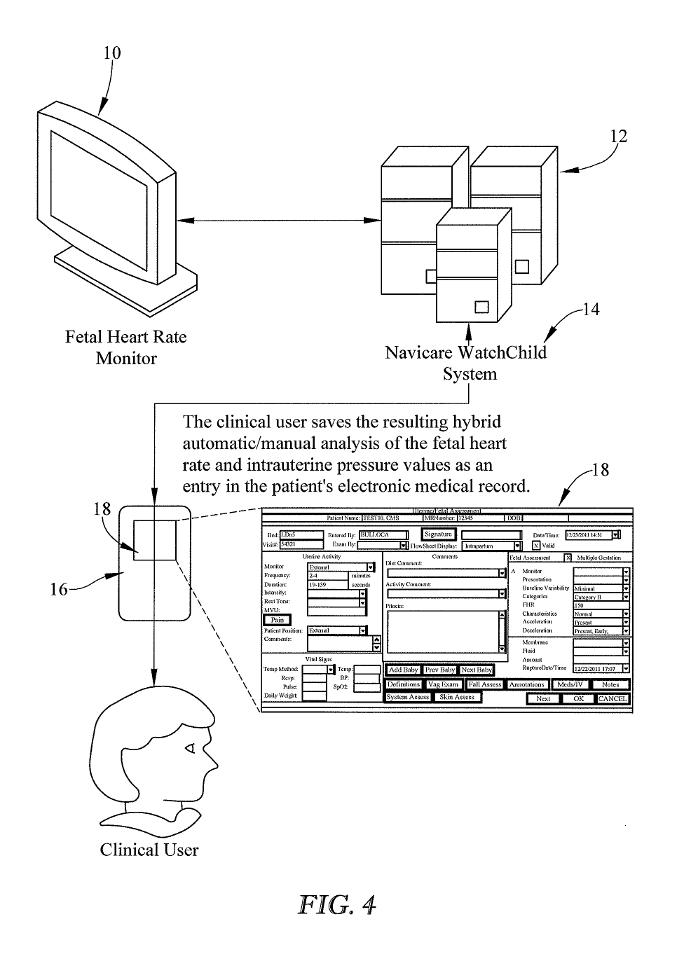

FIG. 3 shows the composite manual and automatic fetal analysis system of FIGS. 1 and 2 wherein the software program 14 is configured to allow a user to manually edit the displayed patterns via the user interface 16. FIG. 4 shows the system of FIGS. 1-3 wherein the software program 14 is configured to allow a user to save the edited composite manual and automatic fetal heart rate and uterine pressure data as an entry into an electronic record of the patient.

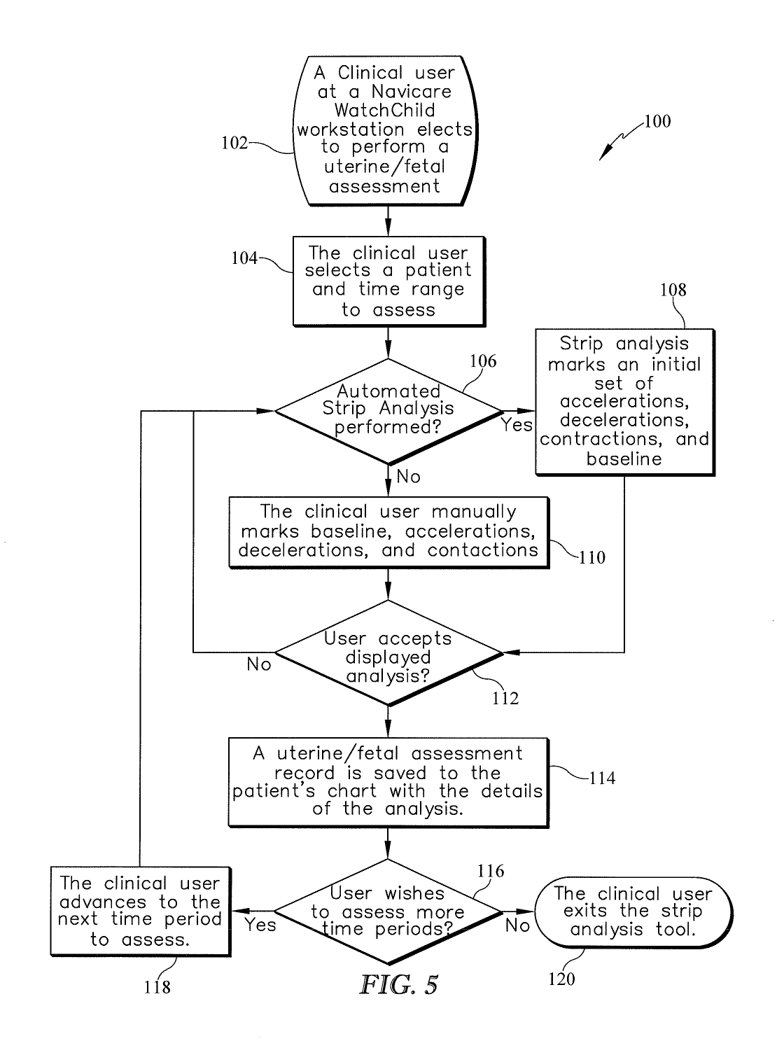

The flowchart 100 shown in FIG. 5 shows one embodiment of a method of use of the composite manual and automatic fetal analysis systems. In operation 102 a user elects to perform a uterine/fetal assessment accessing software program 14 via user interface 16. In operation 104 the user selects a patient and time range to analyze. In operation 106 if the user initiates automated analysis, then in operation 108 the software program 14 indicates at least one of accelerations, decelerations, contractions and baseline values on display 18. If automated analysis is not initiated in operation 106, the user manually marks at least one of accelerations, decelerations, contractions and baseline values in operation 110.

After completion of operations 108 and/or 110, the user makes a decision whether or not to accept the displayed results in operation 112. If the user accepts the displayed results in operation 112, a uterine/fetal assessment record is saved to the patient's medical record. In operation 116 the user makes a decision whether or not to assess more time periods. If the user does not wish to assess more time periods in operation 120 the user may exit the analysis tool. If in operation 116 the user wishes to assess more time periods, the user selects a time period for assessment in operation 118 at which point operation 106 is performed. If during operation 112 the user does not accept the results displayed, operation 106 is repeated.

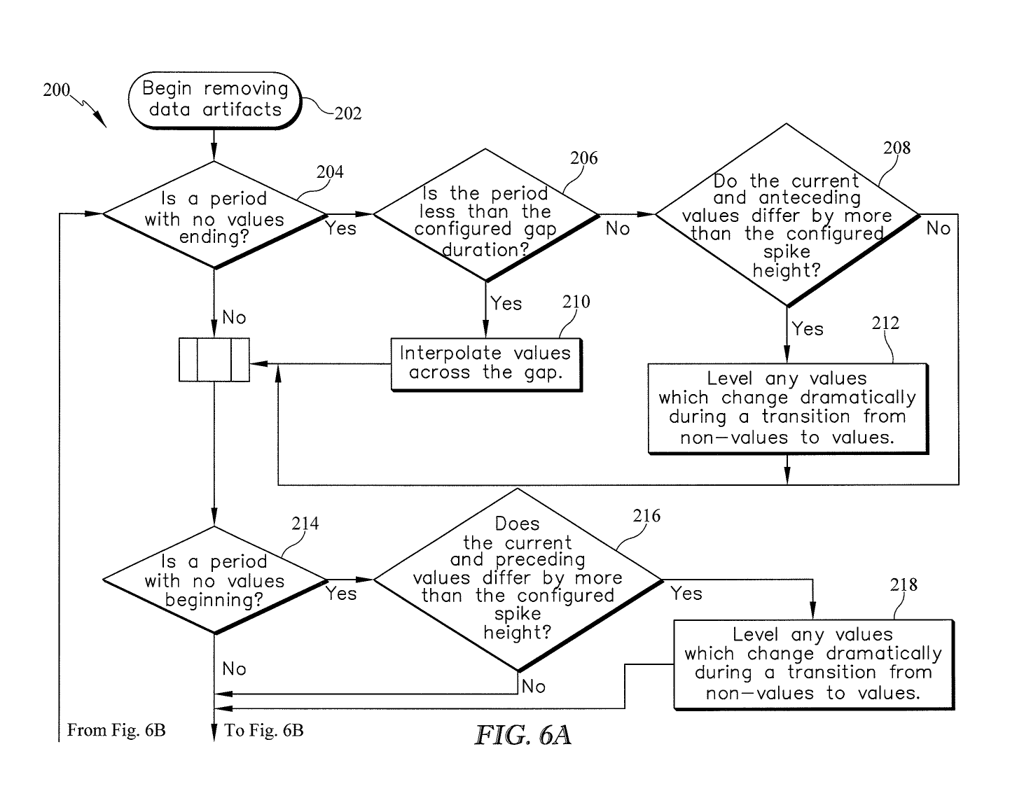

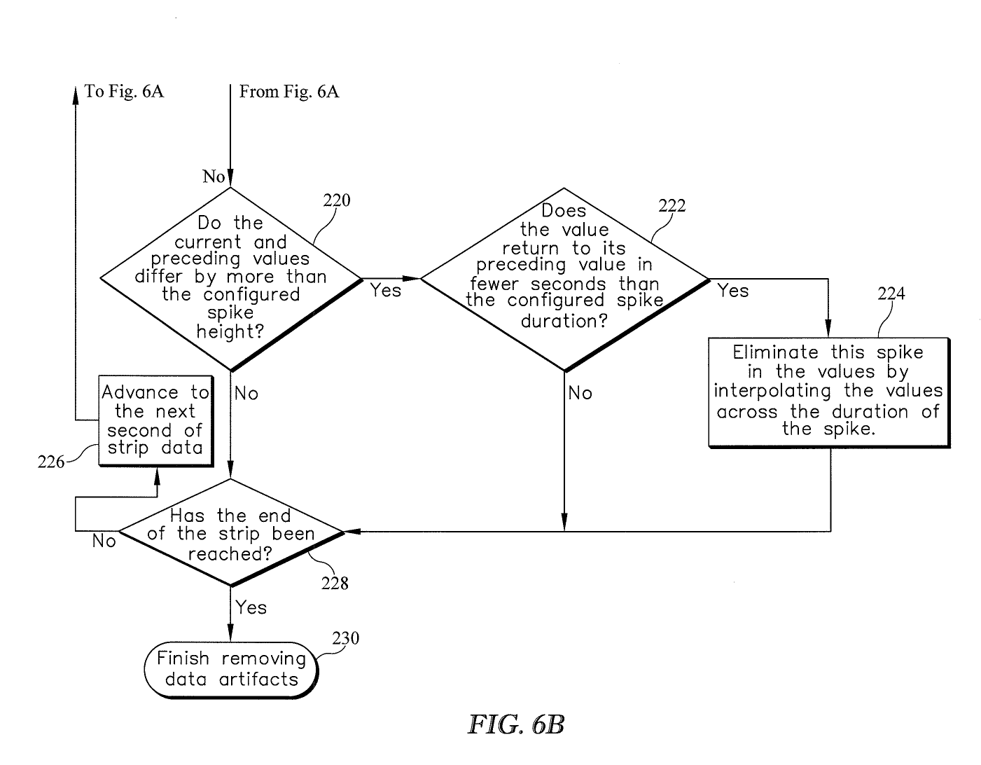

Flowchart 200 shown in FIGS. 6A and 6B shows one embodiment showing an analysis performed by a composite manual and automated fetal analysis system to identify and interpolate gaps in data. In one embodiment the National Institute of Child Health and Human Development (NICHD) characteristics of the time segment selected by the user are calculated from the conditioned data obtained by applying the process shown in flowchart 200 to data for time segment selected by the user and received from the fetal heart rate monitor 10. Prior to performing an automated analysis of patterns and characteristics in fetal heart rate and uterine pressure signals, a pre-processing operation shown in flowchart 200 is performed in this embodiment in order to remove the errant artifacts that may, in one embodiment, be introduced by poor probe connections. These artifacts typically take the form of either gaps (brief periods of time in which the fetal monitor 10 reports no value) or spikes (brief periods of time in which the fetal heart rate or uterine pressure increases or decreases too sharply to be attributed to physiological causes) in the signal.

This artifact elimination process is performed independently for the fetal heart rate and uterine pressure signals in this embodiment. In this embodiment the potential artifacts for each signal type are parameterized by three configurable values: the maximum gap duration, the maximum spike duration, and the minimum spike height (in either beats per minute or mmHg, respective to the signal type).

The algorithm for eliminating artifacts from strips proceeds through all of the data samples in the assessment time period in ascending chronological order, applying the following criteria:

If the current sample has a value and the previous sample had no value, it is considered the end of a potential gap in the strip. If this period with no values has a duration which is less than the maximum gap duration configured for this signal type, then this period is considered a gap artifact, and this gap is eliminated by linearly interpolating the values across this gap. This interpolation is performed between the last sample with a value and the current sample. If the period without values is greater in duration than the configured maximum gap duration for this signal type, then this period cannot be considered a gap artifact, and is instead considered a period with no signal. The transition from an absence of signal to a presence of signal is evaluated for a sudden rise or fall in value which exceeds the minimum spike height in severity. In this case, these initial samples with values are normalized in order to remove the artifact.

If the current sample has no value and the preceding sample has a value, it is considered the beginning of a potential gap in the strip. The transition from a presence of signal to an absence of signal is evaluated for a sudden rise or fall in value which exceeds the minimum spike height in severity. In this case, these final samples with values are normalized in order to remove the artifact.

In operation 202 the process or removal of data artifacts is initiated. In operation 204 a determination is made as to whether a period with no values is ending. If the answer to the query in operation 204 is yes, in operation 206 a determination is made if the period is less than the configured gap duration. If answer to the query in operation 206 is no, a determination is made in operation 208 whether the current and anteceding values differ by more than the configured spike height. If the answer to the query in operation 206 is yes, values are interpolated across the gap in operation 210. If the answer to the query in operation 208 is yes, any values which change dramatically during a transition from non-values to values are leveled in operation 212. If the answer to the query in operation 208 is no and/or after operation 210, and/or after operation 212, a determination is made whether a period with no values is beginning in operation 214. If the answer to the query in operation 214 is yes a determination is made whether the current and preceding values differ by more than the configured spike height in operation 216. If the answer to the query in operation 216 is yes, any values which change dramatically during a transition from non-values to values are leveled in operation 218. If the answer to the query in operation 216 is no and/or after operation 218, a determination is made as to whether the current and preceding values differ by more than the configured spike height in operation 220. If the answer to the query in operation 220 is yes, a determination is made in operation 222 whether the value returns to its preceding value in fewer seconds than the configured spike duration. If the answer to the query in operation 222 is yes, this spike if eliminated by interpolating values across the duration of the spike in operation 224. If the answer to the query in operation 220 is no and/or the answer to the query in operation 222 is no and/or after operation 224 a determination is made whether the end of the strip has been reached in operation 228. If the answer to the query in operation 228 is yes, an operation to finish the process of removing artifacts is performed in operation 230. If the answer to the query in operation 228 is no, analysis advances to the next second of the time period selected for analysis in operation 226. After completion of operation 226, the analysis goes to operation 204.

If the current and preceding samples differ in value by more than the minimum spike height, it is considered the beginning of a potential spike artifact. The duration of this potential spike artifact is evaluated by measuring the duration it takes the ensuing data samples to return to its initial value. If this duration is less than the maximum spike duration, then the region is considered a spike artifact, and it is eliminated by linearly interpolating the values across the duration of the spike.

A user manual for the strip analysis functionality of the The NaviCare® WatchChild® System was included in U.S. Provisional Patent Application No. 61/715,896 to which the present application claims benefit and which is already incorporated by reference herein. Accordingly, the user manual of The NaviCare® WatchChild® System is incorporated by reference herein. The user manual describes the subject matter herein and teaches embodiments of composite manual and automated fetal analysis systems and methods of use in a non-limiting manner.

The use of the terms “a” and “an” and “the” and similar referents in the context of describing the subject matter (particularly in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Furthermore, the foregoing description is for the purpose of illustration only, and not for the purpose of limitation, as the scope of protection sought is defined by the claims as set forth hereinafter together with any equivalents thereof entitled to. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

Preferred embodiments are described herein, including the best mode known to the inventor for carrying out the claimed subject matter. Of course, variations of those preferred embodiments will become apparent to those of ordinary skill in the art upon reading the foregoing description. The inventor expects skilled artisans to employ such variations as appropriate, and the inventor intends for the claimed subject matter to be practiced otherwise than as specifically described herein. Accordingly, this claimed subject matter includes all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

The disclosures of any references and publications cited above are expressly incorporated by reference in their entireties to the same extent as if each were incorporated by reference individually.

1. A system comprising:
a computer comprising a software program configured to normalize gaps in at least one of a signal indicative of a physiological parameter of a fetus and a signal indicative of uterine activity; and
a display whereon said software program is configured to display at least one of said signal indicative of said physiological parameter of a fetus and said signal indicative of uterine activity.

2. The system of claim 1, further comprising a user interface configured to allow a user to choose a time segment of at least one of said signal indicative of said physiological parameter of a fetus and said signal indicative of uterine activity for analysis.

3. The system of claim 2, wherein said user interface is configured to allow a user to initiate automatic detection of a pre-determined pattern from at least one of said signal indicative of said physiological parameter of a fetus and said signal indicative of uterine activity.

4. The system of claim 3, wherein said pre-determined pattern is defined by a National Institute of Child Health and Human Development (NICHD) guideline.

5. The system of claim 3 wherein results of said automatic detection are displayed on said display.

6. The system of claim 5 wherein said user interface is configured to allow a user to manually alter said results of said automatic detection.

7. The system of claim 6 wherein said software program is configured to allow a user to save said altered results of said automatic detection in an electronic database.

8. A system comprising:
a fetal monitoring system configured to capture at least one of a signal indicative of said physiological parameter of a fetus and a signal indicative of uterine activity;
a computer configured to communicate with said fetal monitoring system, said computer comprising a software program configured to normalize gaps in at least one of a signal indicative of said physiological parameter of a fetus and a signal indicative of uterine activity;
a display, whereon said software program is configured to display at least one of said signal indicative of said physiological parameter of said fetus and said signal indicative of said uterine activity; and
a user interface configured to communicate with said computer, said user interface configured to allow a user to initiate automatic detection of a pre-determined pattern from at least one of said signal indicative of said physiological parameter of a fetus and said signal indicative of uterine activity.

9. The system of claim 8 wherein said user interface is configured to allow a user to initiate automatic detection of a pre-determined pattern from at least one of said normalized signal indicative of said physiological parameter of a fetus and said normalized signal indicative of uterine activity.

10. The system of claim 8 wherein said user interface is configured to allow a user to manually alter said results of said automatic detection.

11. The system of claim 10 wherein said software program is configured to allow said user to save said altered results of said automatic detection in an electronic database.

12. A system comprising:
means for initiating automatic detection of a pre-determined pattern from at least one of a signal indicative of said physiological parameter of a fetus and a signal indicative of uterine activity;
means for displaying results of said automatic detection of said pre-determined pattern;
means for allowing a user to manually alter said results of said automatic detection; and
means for allowing a user to save said altered results of said automatic detection in an electronic database.
